(19) United States
(12) Patent Application Publication
(10) Pub. No.: US 2023/0049988 A1
(43) Pub. Date: Feb. 16, 2023

(54) FLUORESCENT DYE IN TERNARY COMPLEX

(71) Applicant: DAXOR CORP., New York, NY (US)

(72) Inventors: Jonathan Feldschuh, New York, NY (US); Atilio Anzellotti, Oak Ridge, TN (US); Nancy Tommye Jordan, Knoxville, TN (US); Boyce Lee Muller, Knoxville, TN (US); Robin D. Zimmer, Loudon, TN (US); Adam Michael Cable, Lenoir City, TN (US)

(73) Assignee: DAXOR CORP., New York, NY (US)

(21) Appl. No.: 17/612,238

(22) PCT Filed: Apr. 29, 2021
(86) PCT No.: PCT/US2021/029906

§ 371 Date: Nov. 18, 2021

Related U.S. Application Data
(60) Provisional application No. 63/017,761, filed on Apr. 30, 2020.

(51) Int. Cl. A61K 49/00

(52) U.S. Cl. CPC A61K 49/0056; A61K 49/0034; A61K 49/0089; A61K 49/0043

(57) ABSTRACT

Pharmaceutical compositions and methods are presented for creating a ternary structure involving a fluorescent molecule, an intermediate carrier molecule, and a larger protein or polymer with a binding site receptive to the intermediate molecule or fluorescent/intermediate complex. The resulting ternary system improves the binding stability of the fluorescent dye to the protein, both in-vivo and in-vitro. This improved stability results in a longer half-life in medical use, enabling improved qualitative and quantitative use of the dye.

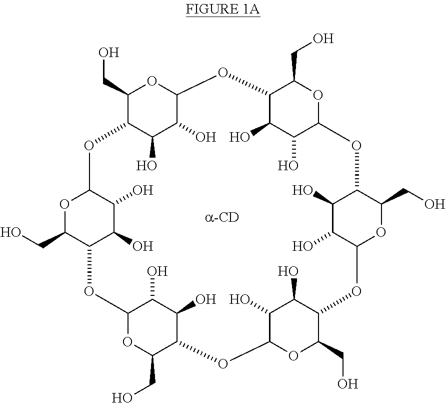

### CROSS-REFERENCE TO RELATED APPLICATIONS

This application claims the benefit of U.S. Provisional Patent Application No. 63/017,761, filed on Apr. 30, 2020, the contents of which are herein incorporated by reference into the subject application.

### FIELD OF THE INVENTION (TECHNICAL FIELD)

The present invention relates to the preparation of supramolecular systems designed to enhance the performance of fluorescent dyes for medical and veterinary use (diagnostic and imaging).

### BACKGROUND

Fluorescent dyes or probes have many medical uses for diagnosis, imaging, and quantitative measurements. Most fluorescent dyes approved for medical use are small molecules which have a relatively short half-life in the body, due to inherent aqueous instability or as they are quickly eliminated from the bloodstream via the kidney and liver. For many applications it is desirable that the effective half-life of a fluorescent probe (FP) be extended to facilitate detection and measurement. While larger fluorescent molecules exist, and it is in general possible to covalently bind fluorescent elements to larger molecules such as proteins, such molecules would require extensive testing for safety and toxicity to receive approval for human use.

Human serum albumin (HSA) is the most abundant protein in plasma with a concentration of 35-50 g/L in serum. This protein is very soluble with a remarkable stability, i.e. it is stable in the pH range 4-9, soluble in ethanol 40% and can be heated to 60° C. for up to 10 h without deleterious effects.

As illustrated in FIG. 2A, HSA consists of 585 amino acids forming a monomeric globular shape, which can be further divided into three α-helical domains. There are three homologous domains named I, II and III, and in turn each domain is known to be made up by two separate helical subdomains A and B, connected by a random coil.

Ligands that are hydrophobic such as warfarin, bilirubin, and non-steroidal anti-inflammatory drugs bind with high affinity to a pocket located in site IIA which is dominated by strong hydrophobic interactions (Sudlow site 1). Ligands with aromatic carboxylates and extended conformation like profens and benzodiazepines bind with high affinity to the polar cationic pocket of site IIIA which involves dipole-dipole, van der Waals and hydrogen bonding type of weak interactions (Sudlow site 2).

In vivo HSA acts as a transport or carrier of many hydrophobic, aromatic and charged compounds via a host-guest interaction on these sites. This type of interaction does not form chemical bonds between the chemical species involved and is formally known as “non-covalent”, this also implies a dynamic equilibrium (i.e. reversible) where the guest or cargo can be delivered or separated upon mild changes in the biological conditions.

In addition to these carrier capabilities, HSA shares very important characteristics with other bio macromolecules, which make ideal as a carrier for a fluorescent marker: non-toxicity, minimal immunogenicity, biocompatibility, biodegradability, long blood circulation time, targeting ability, and water solubility.

Indocyanine green or ICG is an amphiphilic, tricarbocyanine dye, with a net charge of −1 which is usually used as the sodium salt (FIG. 3A). It is an FDA approved dye with a large number of medical applications including retinal angiography, measurement of plasma volume, cardiac output, photocoagulation, assessment of burn depth liver function, and exercise physiology. Its low toxicity and unique optical properties, including its very strong absorption band (780 nm) and effective emission band (800-820 nm), make ICG ideally suited for optical imaging in cells and tissues.

Although ICG is currently the most commonly used fluorescing agent, it has a number of properties that limit its useful for certain applications, especially quantitative ones. ICG injected into a living being displays a tendency to aggregate, rapid degradation in aqueous solution, rapid elimination from circulation, poor photo-stability, and non-specific binding to proteins. These features limit the use of this dye in novel applications such as Photothermal/Photodynamic Tumor Therapy and other time-sensitive surgical procedures; also, notably it could also limit its use for Blood Volume Analysis.

Fluorescein (FLS), is a fluorescent probe used to enhance the visualization of blood or lymph vessels especially in ophthalmology and optometry and is also approved by the FDA (see FIG. 3B). FLS also shares some of the limitations of ICG such as lack of specificity and low fluorescence once injected. Its peak excitation (absorption) at 494 nm and peak emission at 512 nm results in optical properties amenable to tissue imaging.

ICG and FLS do bind non-covalently with HSA. The Sudlow sites are usually preferred according to molecular modeling and fluorescence experiments although other lower affinity sites are possible. These binary complexes of ICG-HSA and FLS-HSA can be achieved by premixing, have some desirable properties in terms of circulation dynamics. They have somewhat longer half-lives in circulation than ICG or FLS alone. However, the binding to HSA is in dynamic equilibrium, and once ICG-HSA or FLS-HSA enters the bloodstream it is likely that binding to other blood proteins (of which there are approximately 20,000 different types) will occur in unpredictable ways that limit the potential for quantitative measurements.

Cyclodextrins (CDs) are produced by enzymatic degradation of starch and are chemically and physically stable. They share some of the characteristics that were presented previously for HSA as carrier, such as water-solubility, biocompatibility in nature with a hydrophilic outer surface and a lipophilic cavity. They have the shape of a truncated cone or torus rather than a perfect cylinder due to the chair conformation of glucopyranose unit (FIG. 1D). Cyclodextrins are classified as natural and derived, among the former group of natural cyclodextrins three well known industrially produced are α, β, and γ consisting of 6, 7, and 8 glucopyranose units (FIGS. 1A-1C). They are crystalline, homogeneous, and non-hygroscopic substances. Amongst these, β-cyclodextrin (β-CD) is ideal for complexation due to its perfect cavity size, efficient drug complexation and loading, availability, and relative low cost. FIGS. 1A-1C show the structure and conformation of natural cyclodextrins. Various hydrophilic, hydrophobic, and ionic derivatives have been developed and utilized to improve the physicochemical and biopharmaceutical properties of drug and inclusion capacity of natural cyclodextrins. The depth of the cavity is the same for all three while both the top and bottom diameters are increased with the number of glucose units.

Evidence for inclusion or complexation of the fluorescent probes ICG and FLS in CDs have been reported previously. ICG inclusion inside β-CD was reported in 2010 with the formation of 1:1 complexes favored (Barros, T. C. et al.; J Phys Org Chem., 2010, 23(10), 893, hereby incorporated by reference in its entirety into the subject application). In 2015 the inclusion of ICG in β-CD and a commercial modified β-CD with sulfobutyl groups (Captisol®) groups was found to enhance and stabilize the fluorescence of ICG (Sitharaman, B. et al.; J Biomed Mater B Appl Biomater., 2016, 104(7) 1457, hereby incorporated by reference in its entirety into the subject application). There is also evidence for inclusion systems with natural CDs and HSA, with β-CD the stronger ligand driven by entropic and enthalpy factors producing a net stabilizing effect in HSA according to isothermal calorimetry and other spectroscopic experiments.

### SUMMARY OF THE INVENTION

Pharmaceutical compositions and methods are presented for creating a ternary structure involving a fluorescent molecule, a saccharide with a high non-covalent affinity for the fluorescent molecule as an intermediate carrier molecule, and a larger macromolecular carrier such as a protein or polymer with a binding site receptive to the intermediate molecule or fluorescent/intermediate complex. The complex is stabilized by non-covalent forces such as but not limited to H-bonds, π-stacking, hydrophobic interactions, salt-bridges, etc. The resulting ternary system improves the binding stability of the fluorescent dye to the protein, both in-vivo and in-vitro. This improved stability results in a longer half-life in medical use, enabling improved qualitative and quantitative use of the dye.

Given the potential performance issues related to low fluorescence and lack of specificity noted above, it is understandable that attempts to modify the structure of ICG and FLS have been tried; however, since covalent chemical modifications to the probes could complicate the medical application requirements, a non-covalent approach in the form of inclusion complexes is used in the present invention.

The present invention discloses methods for preparing non-covalent ternary complexes of approved molecules, Generally Recognized As Safe (GRAS) by the US FDA. This approach has the advantage of not introducing any new molecules to medical use. This substantially lowers the regulatory burden of proving safety for such complexes. The macromolecular carrier used can have the property of being either a component of the blood of a living being (e.g. serum albumin, plasma globulins) or a macromolecule capable of being tolerated in the blood of a living being (e.g. biodegradable polymers, liposomes or modified polypeptides).

### BRIEF SUMMARY OF EMBODIMENTS OF THE PRESENT INVENTION

In one embodiment, the ternary structure is composed of

a) Fluorescent dye,

b) A saccharide with a high non-covalent affinity for a), and

c) a suitable macromolecular carrier that can harbor a)+b).

In one embodiment, b) is a cyclodextrin. These oligosaccharides have a bowl shape that forms a natural container for small molecules with such as fluorescent dyes. In another embodiment this cyclodextrin is modified (by the addition or modification of groups) to enhance its non-covalent affinity for c)

In another embodiment, a conjugating moiety such as modified N-hydroxysuccinimide or modified maleimide is used to tether b) to c). Such a modification is illustrated in FIGS. 6A and 6B.

In one embodiment, c) is HSA in dimeric form or in a high molecular weight aggregates, such as nanoparticles.

In one embodiment, a) is ICG. In another embodiment, a) is FLS.

In another embodiment, b) is Captisol.

In another embodiment, the molar ratios of a:b:c are 1:B:C, where B and C are chosen with the intent of ensuring that the percentage of a) that appears in the final product in the bound ternary state is close to 100%. This is particularly desirable for applications (such as quantitative measurement) where it is important that the fluorescent molecule stays bound to the large carrier molecule c).

In one embodiment, a precise amount of the pharmaceutical composition is provided in a single-use dispensing device. This facilitates quantitative measurement.

In one embodiment, the pharmaceutical composition is lyophilized into a dried product for convenience of storage, transport, and usable life. In another embodiment, a single-use dispensing device includes a mechanism for precise reconstitution of the lyophilized composition before use. This is achieved, for example, by the provision of a precise amount of suitable solvent (such as water or saline) in a sterile assembly with provision for introducing the dried product to the solvent, mixing the product in the solvent to ensure it is in solution, and then precisely dispensing the product.

In one embodiment, a ternary structure of fluorescent dye, a saccharide with a high non-covalent affinity for the fluorescent dye, and a suitable macromolecular carrier that can harbor the saccharide-fluorescent dye complex is achieved by basic mixing, using the fluorescent dye, saccharide, macromolecular carrier in a suitable molar ratio (such as 1:B:C, where C>B>1), and consisting of a sequential process of mixture, by following a method such as the following:
a. Dissolving the saccharide in saline (or similar solvent),
b. Agitating the resulting solution,
c. Incubating the resulting solution,
d. Dissolving the fluorescent dye in solution and then adding it to the saccharide solution,
e. Agitating the resulting solution,
f. Incubating the resulting solution,
g. Adding a solution of the macromolecular carrier to the resulting solution,
h. Agitating the resulting solution,
i. Incubating the resulting solution,
j. transferring to a suitable container, and
k. storing under suitable light and temperature control.

The molar ratios are chosen to be 1:B:C, where C>B>1, so that dynamic equilibrium of binding favors the ternary complex formation for the majority of ICG molecules.

In another embodiment, the addition of macromolecular carrier solution in step g) is performed with a large excess of fluorescent-saccharide complex relative to the macromolecular carrier, and before step j) a size-exclusion filter is used to remove unbound fluorescent-saccharide complex from the resulting product. In another embodiment, fluorescein is used instead of ICG.

In another embodiment, the fluorescent dye is ICG.

In another embodiment, the saccharide is a cyclodextrin or modified cyclodextrin.

In another embodiment, the macromolecular carrier is HSA. In another embodiment the HSA can be unfolded reversibly using temperature, pH or a chaotropic agent (i.e. ethanol or cholesterol) in order to enhance the inclusion of the ICG-β-CD complex inside the HSA.

In another embodiment a conjugating moiety such as modified N-hydroxysuccinimide or modified maleimide can be used to tether the CD to the protein (FIGS. 6A, 6B). In these figures, R is the protein and R′ is the cyclodextrin. The resulting ternary complex would include non-covalent bonding of the fluorescent tracer with the covalently bonded R—R′ complex.

In one embodiment, the resulting complex is lyophilized for convenience in storage, distribution and usable life.

In one embodiment, the lyophilized product is provided in single-use containers, where the dried compound can be reconstituted just before use.

### BRIEF DESCRIPTION OF THE SEVERAL VIEWS OF THE DRAWINGS

FIG. 1A-1C: Structure of natural cyclodextrins, with α-CD, β-CD, and γ-CD shown respectively.

FIG. 1D: Conformational structure of β-CD. The larger opening of the bowl shape, on the right, is approximately 7.8 angstroms in inner diameter and 15.3 angstroms in outer diameter.

FIG. 2A: Tertiary Structure of HSA showing α-helical domains and drug binding sites.

FIG. 2B: Molecular simulation of β-CD binding to HSA.

FIG. 3A: Structure of ICG.

FIG. 3B: Structure of FLS.

FIG. 4A. Molecular simulation of ICG with relevant distances in angstroms.

FIG. 4B. Molecular simulation of ICG inserted into lipophilic cavity of β-CD.

FIG. 5A. Molecular simulation of β-CD-HSA.

FIG. 5B. Molecular simulation of ICG-β-CD-HSA.

FIG. 6A. Example of protein bound covalently to cyclodextrin through the use of N-hydroxysuccinimide.

FIG. 6B. Example of protein bound covalently to cyclodextrin through the use of modified maleimide.

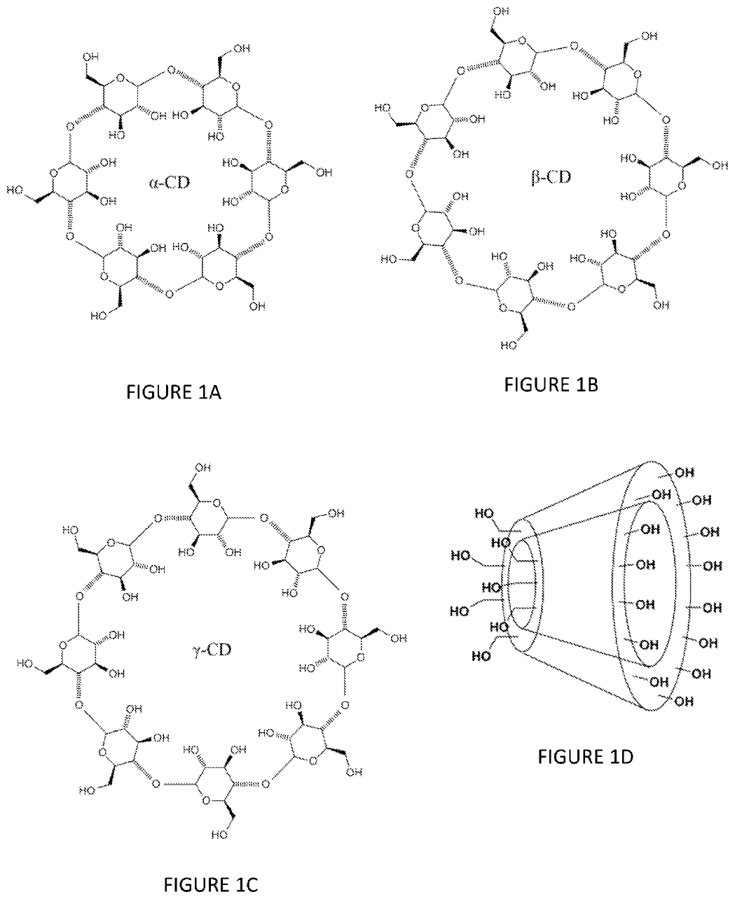

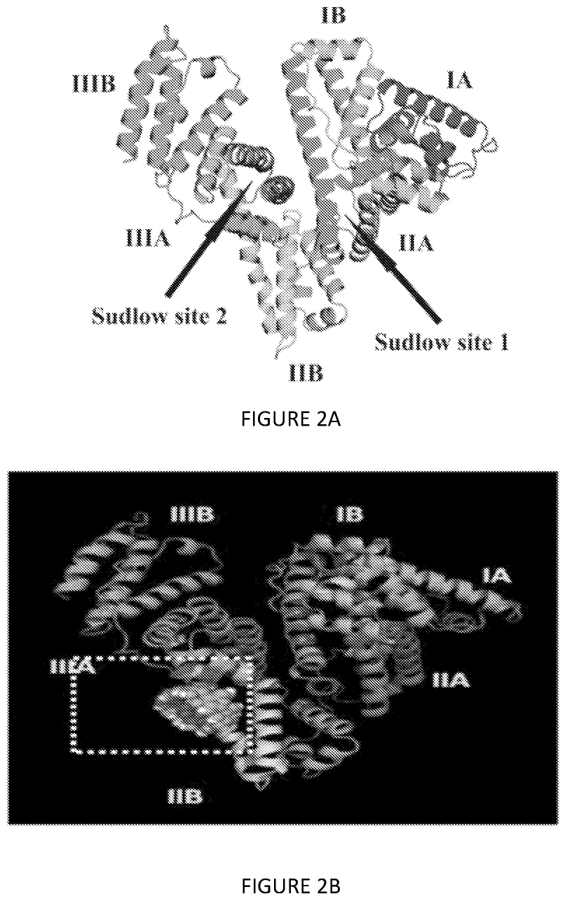

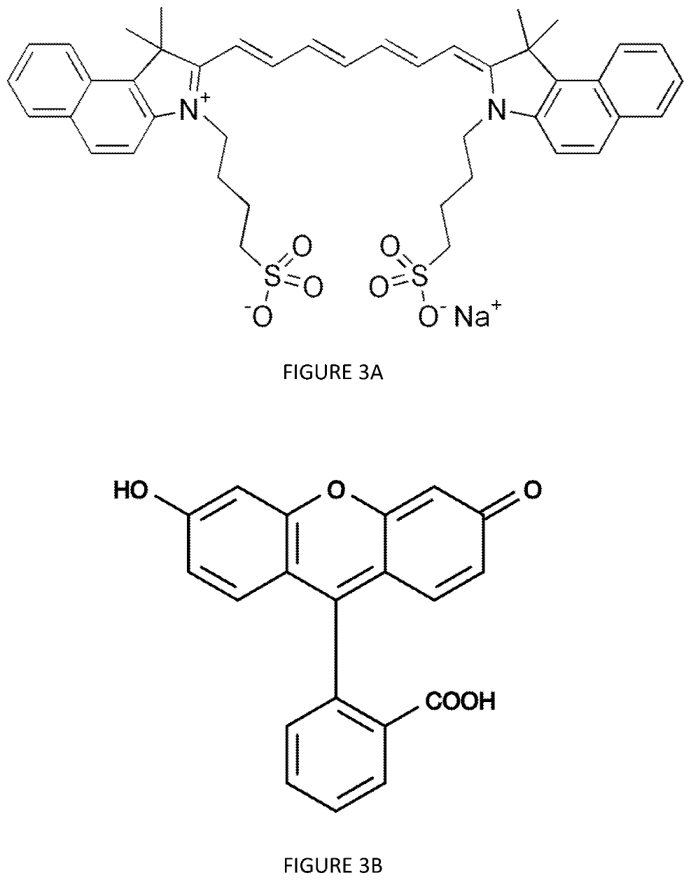

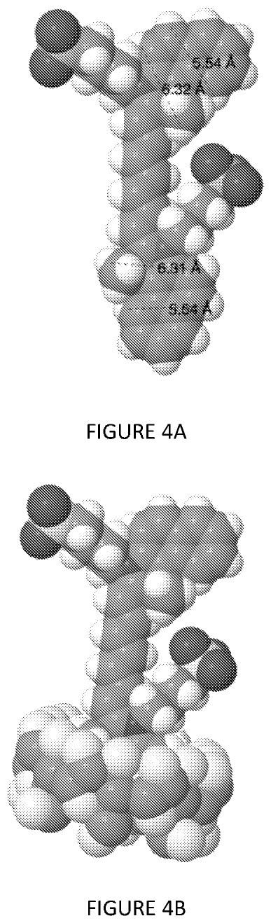

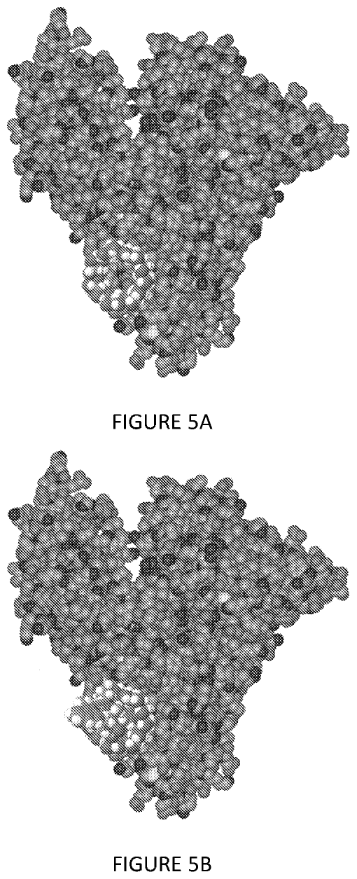

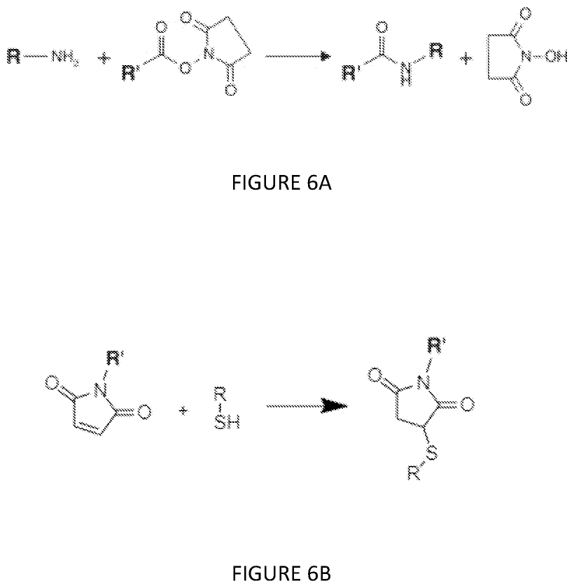

### DETAILED DESCRIPTION OF THE INVENTION

The ternary structure described above can be achieved by basic mixing, using the fluorescent, cyclodextrin, and HSA in a suitable molar ratio (such as 1:1:1), by introducing the components in an appropriate sequence under appropriate conditions. The following is one such procedure. One skilled in the art would recognize variations in this procedure that would also achieve the desired structure.
a. 50 mg Captisol (Cydex's NC-04A-170167TS69) is dissolved in 1.0 ml Normal Saline, and
b. subjected to vortex agitation for 3 minutes at full speed, and then
c. incubated 15 minutes at normal room temperature.
d. 25 mg Indocyanine Green (ICG, Cardiogreen Sigma 12633-100 mg) is rapidly dissolved in weigh boat with 0.3 ml distilled water pipetting up/down for 2 minutes and added rapidly to vial containing Captisol solution, and
e. subjected to vortex agitation for 3 minutes at full speed, and then
f. incubated 15 minutes at normal room temperature.
g. 5 ml of 200 mg/ml Human Serum albumin, (Millipore-Sigma A3782—Fatty acid free, globulin free >99%) solution is added rapidly to the resulting solution of step f) and
h. subjected to vortex agitation for 3 minutes at full speed, and then
i. incubated 15 minutes at normal room temperature,
j. transferred into sterile amber container, and
k. Stored until use at 4-8° C.

In another embodiment, a large excess of HSA is used in step g), so that the molar ratios of fluorescent:CD:HSA are 1:1:N, where N>>1. This ensures that all HSA will be labelled. In this embodiment, in step j) the solution is sterile filtered through a size-exclusion filter such as a 0.2 um cellulose acetate syringe into the sterile amber container to remove excess ICG-CD complex that is unbound to HSA.

The product from step k) can be used directly or lyophilized into a dried product for convenience of storage, transport, and usable life.

For convenience in performance of indicator-dilution volume determinations, the product can be provided in precise quantities in a device capable of delivering the full quantity of the product, such as the Daxor Max-100 syringe.

Formation of the ternary complex can be confirmed and monitored by size-exclusion high-performance liquid chromatography (SEC-HPLC) coupled with a fluorescent detector. The ternary complex exhibiting the fluorescence eluting very close to the retention time of monomeric HSA. Stability of ICG fluorescence can be compared to free ICG in solution to verify increased performance.

The use of cyclodextrins in binary inclusion complexes to make drugs more soluble and modify their pharmacologic properties is widely known. A novel ternary inclusion system comprising A) the fluorescent probe inside B) the cyclodextrin and this inclusion complex inside C) Human Serum Albumin provides benefits from both known binary complexes: the stabilization and solubility benefits of CD-FP, and the preferential, stable binding of CD-HSA. The stable non-covalent of β-CD-HSA yields desirable properties for use in injection, particularly for quantitative measurements. The stability of FP-β-CD ensures that FP present in the system will be primarily in this bound state. The creation of the ternary complex ensures that FP will be stable and preferentially bound to HSA before injection. In addition the stoichiometric nature of the chemical specie will be known, this is worth noting since in many of the applications reported in the literature there is no certainty about the true composition of the chemical specie involved in the application, for instance mixtures of free FP and protein could exist or different loads of FP per protein can lead to ambiguous and non-reproducible results.

HSA starts denaturing reversibly for temperatures of up to 50° C. in a KCl 0.2 M buffer. The inclusion of ICG/CD could be achieved under a specific range of stirring and time, but the process can lead to aggregation if conditions are not controlled, i.e. above 65° C.—this phenomenon can be followed by SEC-HPLC. This is not necessarily a problem since HSA aggregates are non-toxic and have medical applications, e.g. perfusion scintigraphy with 99mTc-HSA. Changes in the 3D structure of HSA can be monitored via UV-vis absorption at 275 nm or circular dichroism.

HSA undergoes transformation and occurs in different isoforms (E: pH 2.6, F: pH 3.4, N: pH 5.6, B: pH 9.4, A). The molecule is stable from low pHs around 2 to 7. Between 7 and 9 a reversible unfolding occurs which can be helpful for non-covalent binding, however after pH of 10 there is a large change in the secondary and tertiary structure of HSA changes, causing its unfolding and an increase in the β-plated sheets, replacing α-helical structure that is generally irreversible (with degradation products such as fragments or aggregates that can be followed by SEC-H PLC).

HSA complexation can be facilitated with chaotropic agents. Concentration of ethanol below 40% v/v are recommended to avoid the formation of aggregates or fibrils. HSA can be reversibly unfolded using a 2-3 M solution of Guanidine HCl as long as the temperature is kept below 30° C.

The N-hydroxysuccinimide (NHS) group is a known conjugating agent to the lysine residue in proteins in general. Another option to couple small molecules to proteins is to take advantage of the maleimide reactivity, which targets cysteines residues specifically. HSA contains 35 cysteine residues, and all of them except one, Cys34 (in domain I), are involved in disulfide bonds stabilizing the structure of HSA; in this way this approach to conjugation can target a fixed location on the protein.

What is claimed is:

1. A pharmaceutical composition of a ternary or three-separate molecules, in a specified range of molar ratios, that interact via non-covalent forces such as but not limited to H-bonds, π-stacking, hydrophobic interactions, salt-bridges, etc. in which the individual components are:
a) a fluorescent dye,
b) a saccharide with a high non-covalent affinity for a), and
c) a suitable macromolecular carrier that can harbor a)+b).

2. The pharmaceutical composition of claim 1, wherein c) having the property of being either a component of the blood of a living being (e.g. serum albumin, plasma globulins) or a macromolecule capable of being tolerated in the blood of a living being (e.g. biodegradable polymers, liposomes or modified polypeptides).

3. The pharmaceutical composition of claim 1, wherein b) is a cyclodextrin.

4. The pharmaceutical composition of claim 1, wherein b) is a cyclodextrin which has been modified to enhance its binding affinity for c).

5. The pharmaceutical composition of claim 1, wherein c) is human serum albumin (HSA).

6. The pharmaceutical composition of claim 1, wherein a conjugating moiety such as modified N-hydroxysuccinimide or modified maleimide is used to tether b) to c).

7. The pharmaceutical composition of claim 4, wherein the composition comprises HSA in dimeric form or in a high molecular weight aggregates, such as nanoparticles.

8. The pharmaceutical composition of claim 1, wherein the fluorescent dye is ICG.

9. The pharmaceutical composition of claim 1, wherein the fluorescent dye is FLS.

10. The pharmaceutical composition of claim 1, wherein b) is Captisol.

11. The pharmaceutical composition of claim 1, wherein the molar ratios of a:b:c are 1:B:C, where B and C are chosen with the intent of ensuring that the percentage of a) that appears in the final product in the bound ternary state is close to 100%.

12. The pharmaceutical composition of claim 1, wherein a precise amount of the composition is provided in a single-use dispensing device.

13. The pharmaceutical composition of claim 1, lyophilized into a dried product for convenience of storage, transport, and usable life.

14. The pharmaceutical composition of claim 13, wherein the single-use dispensing device includes a mechanism for precise reconstitution of the lyophilized composition of before use.

15. A method for preparing the pharmaceutical composition of claim 1, using the fluorescent dye, saccharide, macromolecular carrier in a suitable molar ratio (such as 1:B:C, where C>B>1), and consisting of a sequential process of mixture, such as the steps of
a. dissolving the saccharide in saline (or similar solvent),
b. agitating the resulting solution,
c. incubating the resulting solution,
d. dissolving the fluorescent dye in solution and then adding it to the saccharide solution,
e. agitating the resulting solution,
f. incubating the resulting solution,
g. adding a solution of the macromolecular carrier to the resulting solution,
h. agitating the resulting solution,
i. incubating the resulting solution,
j. transferring to a suitable container, and
k. storing under suitable light and temperature control.

16. The method of claim 15, wherein the addition of macromolecular carrier solution in step g) is performed with a large excess of fluorescent-saccharide complex relative to the macromolecular carrier, and before step j) a size-exclusion filter is used to remove unbound fluorescent-saccharide complex from the resulting product.

17. The method of claim 15, where the fluorescent dye is ICG.

18. The method of claim 15, where the saccharide is a cyclodextrin or modified cyclodextrin.

19. The method of claim 15, where the macromolecular carrier is HSA.

20. The method of claim 19, wherein in steps g) through i) the HSA protein structure is unfolded reversibly using temperature, pH or a chaotropic agent (e.g. ethanol or cholesterol) in order to enhance the inclusion of the fluorescent-saccharide complex inside the HSA.

21. The method of claim 15, wherein a conjugating moiety such as modified N-hydroxysuccinimide or modified maleimide is used to tether the fluorescent-saccharide complex to the macromolecular carrier.

22. The method of claim 15, where the final product of the method is lyophilized into a dried product for convenience of storage, transport, and usable life.

23. The method of claim 15, where the final product of the method is provided in a single-use dispensing device.
